SEQUENCE LISTING (33 sequences)

SEQ ID NO: 1 - RNA, 21 nt, artificial
  siRNA 1 against p65/RelA
ggauugaggagaaacguaauu

SEQ ID NO: 2 - RNA, 22 nt, artificial
  siRNA 2 rev. against p65/RelA
nuuuccuacaagcucguggguu

SEQ ID NO: 3 - RNA, 21 nt, artificial
  siRNA 3 - against p65/RelA
cccacgagcuuguaggaaauu

SEQ ID NO: 4 - RNA, 22 nt, artificial
  siRNA 4 - rev - against p65/RelA
nuuuccuacaagcucguggguu

SEQ ID NO: 5 - RNA, 21 nt, artificial
  siRNA 5 - against p65/RelA
ggcuauaacucgccuaguguu

SEQ ID NO: 6 - RNA, 22 nt, artificial
  siRNA 6 - rev. against p65/RelA
ncacuaggcgaguuauagccuu

SEQ ID NO: 7 - RNA, 21 nt, artificial
  siRNA 7 - against p65/RelA
ccacacaacugagcccauguu

SEQ ID NO: 8 - RNA, 22 nt, artificial
  siRNA 8 - rev- against p65/RelA
ncaugggcucaguugugugguu

SEQ ID NO: 9 - RNA, 19 nt, artificial
  siRNA - control sequence
auguauuggccuguauuag

SEQ ID NO: 10 - protein, 461 aa, artificial
  DN30 heavy chain
MGWSYIILFLVATATDGHSQVQLQQPGTELVKPGASVKLSCKASGYTFTSYWIHWVKQRPGQGLEWIGEINPSSGRTNYNEKFKNKVTVTVDKSSTTAYMQLSNLTSEDSAVYYCASRGYWGQGTTLTVSSAKTTAPSVYPLAPVCGNTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 11 - DNA, 1386 bp, artificial
  DN30 - heavy chain
atgggatggagctatatcatcctctttttggtagcaacagctacagatggccactcccaggtccaactgcagcagcctgggactgaactggtgaagcctggggcttcagtgaagctgtcctgcaaggcttctggctacaccttcaccagttactggatacactgggtgaagcagaggcctggacaaggccttgagtggattggagagattaatcctagcagcggtcgtactaactacaacgagaaattcaagaacaaggtcacagtgactgtagacaaatcttccaccacagcctacatgcaactcagcaacctgacatctgaggactctgcggtctattactgtgcaagtaggggctactggggccaaggcaccactctcacagtctcctcagccaaaacaacagccccatcggtctatccactggcccctgtgtgtggaaatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 12 - protein, 8 aa, artificial
  DN30 heavy chain  - CDR-H1
GYTFTSYW

SEQ ID NO: 13 - protein, 8 aa, artificial
  DN30 heavy chain  -  CDR-H2
INPSSGRT

SEQ ID NO: 14 - protein, 5 aa, artificial
  DN30 heavy chain  - CDR-H3
ASRGY

SEQ ID NO: 15 - DNA, 25 bp, artificial
  CDR-H1 nucleotide
ggctacaccttcaccagttactgga

SEQ ID NO: 16 - DNA, 24 bp, artificial
  CDR-H2 nucleotide
attaatcctagcagcggtcgtact

SEQ ID NO: 17 - DNA, 9 bp, artificial
  CDR-H3 nucleotide
gcaagtagg

SEQ ID NO: 18 - protein, 238 aa, artificial
  DN30 light chain
METDTILLWVLLLWVPGSTGDIVLTQSPASLAVSLGQRATISCKASQSVDYDGGSYMSWFQQRPGQPPKLLISAASNLESGIPARFSGSGSGTDFTLNIHPVEEEDVATYYCQQSYEDPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - DNA, 717 bp, artificial
  DN30 light chain
atggagacagacacaatcctgctatgggtgctgctgctctgggttccaggctccactggtgacattgtgctgacccaatctccagcttctttggctgtgtctctagggcagagggccaccatctcctgcaaggccagccaaagtgttgattatgatggtggtagttatatgagttggttccaacagagaccaggacagccacccaaactcctcatctctgctgcatccaatctagaatctgggatcccagccaggtttagtggcagtggctctgggacagacttcaccctcaatatccatcctgtggaggaggaggatgttgcaacctattactgtcagcaaagttatgaggatccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatctacttcacccattgtcaagagcttcaacaggaatgagtgttag

SEQ ID NO: 20 - protein, 10 aa, artificial
  DN30 light chain  -  CDR-L1
QSVDYDGGSY

SEQ ID NO: 21 - protein, 3 aa, artificial
  DN30 light chain  - CDR-L2
AAS

SEQ ID NO: 22 - protein, 9 aa, artificial
  DN30 light chain  - CDR-L3
QQSYEDPLT

SEQ ID NO: 23 - DNA, 31 bp, artificial
  CDR-L1 nucleotide
aaagtgttgattatgatggtggtagttatat

SEQ ID NO: 24 - DNA, 9 bp, artificial
  CDR-L2 nucleotide
gctgcatcc

SEQ ID NO: 25 - DNA, 27 bp, artificial
  CDR-L3 nucleotide
cagcaaagttatgaggatccgctcacg

SEQ ID NO: 26 - DNA, 21 bp, artificial
  primer NFKBIA
gaaccccagctcagggtttag

SEQ ID NO: 27 - DNA, 20 bp, artificial
  primer rev. NFKBIA
gggaatttccaagccagtca

SEQ ID NO: 28 - DNA, 21 bp, artificial
  primer kB1
aggcccagtgccttattacca

SEQ ID NO: 29 - DNA, 19 bp, artificial
  primer kB1 rev.
gcggcctgactggagattt

SEQ ID NO: 30 - DNA, 24 bp, artificial
  primer kB2
gggactcagtttctttacctgcaa

SEQ ID NO: 31 - DNA, 24 bp, artificial
  primer kB2 rev.
gggactcagtttctttacctgcaa

SEQ ID NO: 32 - DNA, 199 bp, homo sapiens
atggtgtgaaggacacctgactgggctgaaagctaagttctaactttgcccctcttactaaccagctatgtgactctcctgggaacttttagggactcagtttctttacctgcaaaatggttcaatgcaagactttagtaacgtaatgggaactttccttttccataaaactggggaatcaagaggtaatctcttttga

SEQ ID NO: 33 - DNA, 111 bp, mus musculus
cgtacgggctgttttattcatctgcaaaatgattccgtgcaggcctccaaaactgtaataggaactttccttttccatcaaactgaggagtggtgaggtaaaccgctcttg

(19) United States
(12) Patent Application Publication
(10) Pub. No.: US 2012/0237524 A1
(43) Pub. Date: Sep. 20, 2012

(54) MET INHIBITORS FOR ENHANCING RADIOTHERAPY EFFICACY

(76) Inventors: Carla Boccaccio, Torino (IT); Paolo Maria Comoglio, Torino (IT); Fiorella Petronzelli, Roma (IT); Rita De Santis, Pomezia (Roma) (IT)

(21) Appl. No.: 13/423,830

(22) Filed: Mar. 19, 2012

(30) Foreign Application Priority Data
Mar. 18, 2011 (EP) 11158861.2

(51) Int. Cl. A61K 39/395; A61P 35/00; A61K 48/00

(52) U.S. Cl. 424/143.1; 514/44 R

(57) ABSTRACT

Met inhibitor and/or nucleotide sequence encoding a Met inhibitor for use in the treatment of patients suffering from a cancer for reducing and/or abrogating patients' resistance to radiotherapy, wherein the Met inhibitor is selected among: i) an anti-Met monoclonal antibody, ii) a genetically engineered antibody containing the complementarity determining regions (CDRs) of the anti-Met monoclonal antibody, and iii) a fragment of (i) or (ii) containing the complementarity determining regions (CDRs) of the anti-Met monoclonal antibody, or combinations thereof.

### FIELD OF THE INVENTION

The present disclosure concerns the use of MET inhibitors for enhancing the efficacy of radiotherapy in patients suffering from cancers.

### TECHNICAL BACKGROUND

Although successfully employed to treat cancer patients, radiotherapy can fail to eradicate the tumour, which relapses with a more aggressive phenotype. Consistently, a paradoxical pro-metastatic effect of ionizing radiation (IR) has been unveiled by classical studies in animal models. Tumour progression after radiotherapy could result from positive selection of the “cancer stem cell” subpopulation, which is intrinsically radioresistant. However, striking evidence indicates that, aside from selection, IR promotes an adaptive phenotype aimed at tissue regeneration, which can turn out in metastatic behaviour. This phenotype is defined as the “stress-and-recovery” response to DNA damage, occurring both at the single cell and tissue level. In single cells, detection of DNA damage elicits specific molecular mechanisms, mostly orchestrated by the ATM-p53 axis, which block replication and activate DNA repair. If the damage is irreversible, a normal cell is programmed to execute apoptosis, or to hibernate its proliferative ability through senescence. However, after death of mutant cells, tissues must restore an adequate cell number and pattern, so as to recover the original structure and function. Thus, regeneration (or “wound healing”) is initiated by surviving cells, either normal or neoplastic. As observed in vitro, this process includes steps such as detachment from the wound border, acquisition of a fibroblast phenotype, migration into the scratched area, and, possibly, proliferation. The entire program has been referred to as “epithelial-mesenchymal transition” (EMT), a terminology underscoring morphological features. More recently, this program has also been defined as “invasive growth” (IG), a wording that emphasizes functional aspects relevant for cancer. It is now widely accepted that EMT/IG is a physiological program for tissue development and regeneration, which is usurped by cancer cells to perform invasion and metastasis. EMT/IG is activated in cancer cells (a) sometimes, as result of genetic lesions supporting clonal selection; (b) more often, as result of an adaptive response to adverse environmental conditions.

Thus, EMT/IG is a genetic program ultimately controlled by a few specific transcription factors, and orchestrated by a handful of extracellular signals. The latter include scatter factors, such as Hepatocyte Growth Factor (HGF) and Macrophage Stimulating Protein (MSP), which bind tyrosine kinase receptors belonging to the Met family.

### OBJECT AND SUMMARY OF THE INVENTION

The need is therefore felt for improved solutions for enhancing efficacy of radiotherapy in patients suffering from tumors.

The object of this disclosure is providing such improved solutions.

According to the invention, the above object is achieved thanks to the subject matter recalled specifically in the ensuing claims, which are understood as forming an integral part of this disclosure.

An embodiment of the invention provides the use of a Met inhibitor in the treatment of a patient suffering from a tumor, preferably a tumor presenting a deregulated Met pathway, wherein the Met inhibitor is selected from:

i) an anti-Met monoclonal antibody,

ii) a genetically engineered antibody containing the complementarity determining regions (CDRs) of the anti-Met monoclonal antibody, and

iii) a fragment of (i) or (ii) containing the complementarity determining regions (CDRs) of the anti-Met monoclonal antibody, or combinations thereof, wherein the Met inhibitor is able to induce down-regulation of the receptor encoded by the MET gene and reduces and/or abrogates patient's resistance to radiotherapy.

In a preferred embodiment the anti-Met monoclonal antibody is DN30 anti-Met monoclonal antibody produced by the hybridoma cell line ICLC PD 05006.

In a further preferred embodiment the complementarity determining regions (CDRs) contained in a) the genetically engineered antibody or b) the fragments of the anti-Met monoclonal antibody or of the genetically engineered antibody are the CDRs of DN30 anti-Met monoclonal antibody whose amino acid sequences are set forth in SEQ ID No.: 12 to 14 and 20 to 22.

Another embodiment of the present disclosure concerns a nucleotide sequence encoding a Met inhibitor for use in the treatment (e.g. by gene-therapy) of a patient suffering from a tumor, preferably a tumor presenting a deregulated Met pathway, said Met inhibitor being selected from:

i) an anti-Met monoclonal antibody,

ii) a genetically engineered antibody containing the complementarity determining regions (CDRs) of the anti-Met monoclonal antibody, and

iii) a fragment of (i) or (ii) containing the complementarity determining regions (CDRs) of the anti-Met monoclonal antibody, or combinations thereof,

wherein said Met inhibitor is able to induce down-regulation of the receptor encoded by the MET gene and reduces and/or abrogates patient's resistance to radiotherapy.

In a preferred embodiment the anti-Met monoclonal antibody is DN30 anti-Met monoclonal antibody produced by the hybridoma cell line ICLC PD 05006.

In a preferred embodiment the complementarity determining regions (CDRs) contained in the nucleotide sequences encoding a) the genetically engineered antibody or b) the fragments of the anti-Met monoclonal antibody or of the genetically engineered antibody are the CDRs of DN30 anti-Met monoclonal antibody whose amino acid sequences are set forth in SEQ ID No.: 12 to 14 and 20 to 22.

According to a preferred embodiment, the Met inhibitor is for administration i) in the form of soluble protein by injection or infusion or ii) by means of a vector for systemic or intra-tumor administration.

According to a further preferred embodiment, the Met inhibitor is in form of a Fab fragment optionally conjugated with at least one stabilizing molecule, wherein the stabilizing molecule is selected from polyethylenglycol, albumin binding domain, albumin.

The present disclosure discloses that irradiation upregulates MET expression (oncogene known to drive “invasive growth” of cancer), which in turn promotes cell invasion and protects cells from radiation-induced apoptosis. Thus, abrogation of MET expression or inhibition of its kinase activity by specific compounds, i.e. specific Met inhibitors, promote apoptosis and counteract radiation-induced invasiveness, thus enhancing efficacy of radiotherapy.

### BRIEF DESCRIPTION OF THE DRAWINGS

The invention will now be described, by way of example only, with reference to the enclosed figures, wherein:

FIG. 1. IR induces MET transcription.

a, Met protein in MDA-MB-435S at the indicated time-points after irradiation (10 Gy). ctrl, Met at time zero. b, Met protein in MDA-MB-435S 12 h after irradiation (1-10 Gy). c, MET transcript in MDA-MB-435S at the indicated time-points after irradiation (10 Gy). d, Luciferase activity driven by the MET promoter (basic, promoterless construct) in MDA-MB-231 at the indicated time-points after irradiation (10 Gy; ctrl, non-irradiated cells). Columns: mean of triplicate analyses of two independent experiments±s.e.m. (* p<0.05, n=6, paired t-test). a.u., arbitrary units.

FIG. 2. IR-induced MET transcription requires NF-κB.

a, Protein nuclear accumulation in MDA-MB-435S analyzed at the indicated time-points after irradiation (10 Gy), or after 24 h culture in hypoxia (1% O₂). ctrl, non-irradiated cells at time zero. b, Chromatin immunoprecipitation in irradiated MDA-MB-231 (10 Gy; ctrl, non irradiated cells). Columns represent the ratio between anti-p65/RelA and nonspecific IgG immunoprecipitation of each NF-κB binding sequence (κB1 or κB2) in the MET promoter (mean±s.e.m. of triplicate analyses). The .NFKBIA (IκBα) promoter was used as positive control. c, MET promoter activity in MDA-MB-231, silenced for p65/RelA expression (siRELA; siCTRL, control), and irradiated (10 Gy; ctrl, non-irradiated cells). Columns represent the ratio between MET promoter-driven and promoterless (basic) luciferase expression (mean of triplicate analyses in three independent experiments±s.e.m). Inset: p65/RelA protein after siRNA transfection. d, Met protein accumulation in MDA-MB-435S silenced for p65/RelA expression (siRELA; siCTRL, control), at the indicated time-points after irradiation (ctrl, non-irradiated cells at time zero).

FIG. 3. IR-induced MET expression requires ATM kinase activation.

Met protein expression, Chk2 phosphorylation (p-Chk2) and p65/RelA nuclear translocation in MDA-MB-435S treated with the ATM kinase inhibitor CGK733, and extracted at the indicated time-points after irradiation. ctrl, non-irradiated cells at time zero.

FIG. 4. IR-induced invasive growth requires Met.

a, Basement membrane invasion by irradiated MDA-MB-231 or U-251 (10 Gy; ctrl, control). Micrographs of transwell filters (10×). b, Aberrant Met-induced branching morphogenesis in irradiated MDA-MB-4355 (10 Gy; ctrl, control), cultured with or without (−) the indicated HGF concentrations. Scale bar: 100 μm.

FIG. 5. Met inhibition sensitizes cells to IR-induced apoptosis and proliferative arrest.

Viability of U-251 irradiated with 10 Gy and/or cultured in the presence of the Fab fragment of the DN30 anti-Met antibody, for 48 h (vehicle: non-irradiated cells). Columns: mean of triplicate analyses of three independent experiments±s.e.m. (* p<0.05, viability significantly reduced with respect to either Fab-DN30 or 10 Gy alone, n=9, paired t-test). Columns: percentage of cells generating clones (mean of triplicate analyses of two independent experiments±s.e.m., * p<0.05, n=6, paired t-test).

FIG. 6. IR induces Met phosphorylation.

Met phosphorylation in MDA-MB-231 treated with HGF (50 nM) and/or IR (10 Gy) Cells were immunoprecipitated with anti-Met antibodies at the indicated time-points and analyzed by western blot with anti-phospho-Tyr antibodies (p-Tyr). Met was shown as control of protein immunoprecipitation. ctrl, cells treated with HGF negative control (see Methods)

FIG. 7 Alignment of mouse and human MET promoter.

The human MET promoter (GenBank accession N°: AF046925) was analyzed with the TRANSFAC 7.0 software (Biobase Biological Database Gmbh, Wolfenbuttel, Germany) for identification of transcription factor binding sites. Two putative NF-κB binding sites (κB1 and κB2) were found. Alignment of the human and mouse (Gene ID: 17295) MET promoter shows that the κB2 site (−1149/−1136 in the human sequence, rectangle) is highly conserved between the two species.

FIG. 8: Nucleic acid (a) and amino acid (b) sequence of DN30 monoclonal antibody heavy chain. The CDR regions are underlined both in the nucleotide and amino acid sequences.

FIG. 9: Nucleic acid (a) and amino acid (b) sequence of DN30 monoclonal antibody light chain. The CDR regions are underlined both in the nucleotide and amino acid sequences.

### DETAILED DESCRIPTION OF EMBODIMENTS OF THE INVENTION

The present invention will now be described in detail in relation to some preferred embodiments by way of non limiting examples.

In the following description, numerous specific details are given to provide a thorough understanding of embodiments. The embodiments can be practiced without one or more of the specific details, or with other methods, components, materials, etc. In other instances, well-known structures, materials, or operations are not shown or described in detail to avoid obscuring aspects of the embodiments.

The headings provided herein are for convenience only and do not interpret the scope or meaning of the embodiments.

Besides damaging intracellular targets, ionizing radiation (mostly through generation of Reactive Oxygen Species) tunes the activity of regulatory molecules, which control the stress-and-recovery biological response.

Transcriptional upregulation of the MET oncogene emerges as a crucial event in this response, resulting in the execution of a pro-survival and regenerative program that counteracts radiation-induced damage. This disclosure shows that IR-induced MET upregulation is controlled by a signal transduction pathway elicited by the protein kinase ATM following detection of DNA lesions. This pathway involves nuclear export of the ATM kinase and release of the transcription factor NF-κB from inhibition. Remarkably, it is known that activation of NF-κB by DNA damage plays a key role in the defensive response against radiation, as NF-κB is a prominent regulator of anti-apoptotic genes. It has been proposed that cell survival promoted by NF-κB is so effective as to induce “adaptive resistance” of cancer cells to radiotherapy. The present inventors now show that the adaptive response to radiation sustained by NF-κB crucially involves the MET proto-oncogene.

MET induction by IR is a biphasic transcriptional event, mediated by binding of NF-κB to the two KB specific response elements located in the MET promoter. The early transcriptional response occurring within 1-2 h after irradiation likely relies on activation of NF-κB by the intrinsic pathway driven by the DNA damage sensor-ATM. Conceivably, IR-induced Met overexpression is per se sufficient to elicit signal transduction in the presence of physiological concentrations of the ubiquitous ligand HGF, as shown in the case of hypoxia-induced Met overexpression. The late and sustained MET upregulation—prolonged over 24 h—is also likely to be supported by multiple extrinsic signalling pathways impinging on NF-κB. In fact, irradiation promotes expression of cytokines including TNF-α, IL-1 and IL-10 that, on one hand, are NF-κB targets, and, on the other hand, stimulate NF-κB transcriptional activity. The present inventors consider that, in living tissues, irradiation induces autocrine/paracrine loops reverberating on NF-κB that propagate waves of survival signals throughout the damaged tissue.

Remarkably, it is known that the transcriptional response to NF-κB includes, in addition to pro-survival genes, molecules responsible for EMT/IG. The combined execution of pro-survival and EMT/IG genetic programs acts as a double-edge sword: in normal tissues, these programs result in survival and regeneration after damage; in cancer cells, they foster progression towards malignancy.

The MET proto-oncogene meets the criteria for being a critical NF-κB target, required for orchestrating both the bright and the dark side of the stress-and-recovery responses. As it is shown in the instant disclosure, on one hand, IR-induced Met overexpression enables cells to heal wounded monolayers. On the other hand, IR stimulates cells to cross basement membranes, a typical hallmark of malignant tumours. Even more strikingly, it is reported that IR turns the physiological process of Met-induced branching morphogenesis into disorganized cell dissemination throughout a tridimensional matrix. In all cases, although several NF-κB target genes are expressed in irradiated cells, through MET knock-down or functional inhibition, the present inventors show that Met is required for both physiological invasive growth (wound healing) and malignant invasive growth (invasiveness). The reported aggressiveness of tumours relapsing after irradiation may, thus, involve activation of the EMT/IG program under a tight control of the MET oncogene.

The observation that Met is implied in the anti-apoptotic, regenerative and invasive response to IR has important therapeutic consequences: combination of radiotherapy with Met inhibition radiosensitizes cancer cells, while preventing pro-invasive collateral effects. Indeed the present disclosure shows that Met inhibition significantly impairs cell survival and clonogenic ability after exposure to therapeutic doses of IR. Most importantly, being expressed in the stem/progenitor compartment of several normal tissues, MET is conceivably expressed also in cancer stem cells, which often derive from direct transformation of normal stem cells or proliferating progenitors. IR-induced Met expression and activation support cancer (stem) cell radioresistance and invasive ability, thus increasing the chance of their positive selection and dissemination.

Therefore, Met inhibition (by means of administration of the Met inhibitor in form of soluble protein or by gene-therapy i.e. administration of a vector encoding the Met inhibitor as defined in the following) in combination with conventional therapies, i.e. radiotherapy, is a further strategy to eradicate cancer.

In the present disclosure with the expression “Met inhibitor” is meant an anti-Met monoclonal antibody, derivatives and/or fragments thereof able to induce down-regulation of the receptor encoded by the MET gene. In a preferred embodiment the “Met inhibitor” is DN30 anti-Met monoclonal antibody, derivatives and/or fragments thereof which are able to induce down-regulation of the receptor encoded by the MET gene

With the expression “antibody derivative” is meant a molecule containing the Complementary Determining Regions (CDRs) of the antibody, such as a genetically engineered or humanized antibody containing the CDRs of the antibody or a peptide containing the CDRs of the antibody.

With the expression “antibody fragment” is meant a fragment selected from Fv, scFv, Fab, Fab', F(ab′)₂ fragments of i) the anti-Met monoclonal antibody, and ii) genetically engineered or humanized antibody containing the Complementary Determining Regions (CDRs) of the anti-Met monoclonal antibody.

From a pharmacological viewpoint, the employment of a Fab fragment has both advantages and disadvantages. Fab molecules can be easily produced using simple expression systems including lower eukaryotes and prokaryotes (Chambers R S. Curr Opin Chem Biol 2005 9:46-50). Fab molecules are also less immunogenic compared to whole antibodies and their lower molecular weight improves tissue penetration.

A problem in the use of Fab fragments in clinics relates to the short plasma half-life of Fab fragments that is due to higher kidney clearance. This can be circumvented by local administration of the Fab molecule to the tumor site. For therapeutic applications that require systemic delivery and prolonged treatment, actions aimed at incrementing Fab half-life are necessary. In order to get an incremented Fab half-life, a stabilized form of Fab obtained by conjugation with a stabilizing molecule (that does not modify the antigen binding properties of the Fab fragment) has been realized.

Although pegylation is the most consolidated technique (Chapman A P. Adv Drug Deliv Rev 2002 54:531-545.), pegylation is not the only possibility for implementing the stability of therapeutic proteins.

Alternatively to the chemical modification, the recombinant Fab molecules can be modified at the level of primary nucleotide sequence to incorporate sequences encoding peptides or domains capable to bind with high affinity the serum albumin (Dennis M S, et al., J Biol Chem 2002 277:35035-35043; Stork R, et al. Protein Eng Des Sel 2007 20:569-576) or can be generated as a chimeric molecule in which one of the chain encoding the Fab is fused in frame with a sequence encoding a protein biologically inactive (e.g. serum albumin (Subramanian G M, et al. Nat Biotechnol 2007 25:1411-1419)). Polyethylenglycol, albumin binding domain, albumin, or any other sequence that does not modify the antigen binding properties of the Fab fragment can be used as stabilizing molecules capable to increase the in vivo plasma half-life of the Fab fragment.

DN30 anti-cMet monoclonal antibody is produced by the hybridoma cell line deposited by Advanced Biotechnology Center (ABC), Interlab Cell Line Collection (ICLC), S.S. Banca Cellule e Colture in GMP, Largo Rosanna Benzi 10, Genova, Italy with accession number ICLC PD 05006.

Tumors suitable for administration of a Met inhibitor in order to reduce and/or abrogate radiotherapy resistance according to instant disclosure include i) carcinomas, preferably selected between bladder, breast, cholangiocarcinoma, colorectal, endometrial, esophageal, gastric, head and neck, kidney, liver, lung, nasopharyngeal, ovarian, pancreas/gall bladder, prostate, thyroid, ii) soft tissue sarcoma, preferably selected among Kaposi's Sarcoma, Leiomyosarcoma, MFH/Fibrosarcoma, iii) musculoskeletal sarcoma, preferably selected among osteosarcoma, rhabdomyosarcoma, synovial sarcoma, iv) hematopoietic malignancy, preferably selected among acute myelogenous leukemia, adult T cell leukemia, chronic myeloid leukemia, lymphomas, multiple myeloma, v) other neoplasms preferably selected among brain tumors, melanoma, mesothelioma, Wilms' tumor.

All these tumors present, in fact, a “deregulated Met pathway”, wherein this expression means that these tumors are characterized by an aberrant Met signaling due to at least one of i) Met mutations, ii) Met protein overexpression, iii) Met gene amplification, iv) elevated levels of circulating HGF.

Administration of Met Inhibitors to Human Patients

Anti-Met antibodies will be administered through regimens similar to those adopted for antibodies targeting other receptor tyrosine kinases involved in human malignancies (e.g. Trastuzumab, an antibody against HER-2). Typically, the antibody or a derivative or fragment thereof is administered by intravenous infusion with weekly doses ranging between 5-10 mg/kg, preferably 4-8 mg/kg. For combination with radiotherapy, administration of the anti-Met antibodies will start one week, more preferably one day, before irradiation and continue until one week, preferably until 6 to 48 hours, after the end of radiotherapy.

The cDNA sequences encoding the anti-Met antibody, or derivatives or fragments thereof can be also administered to human patients through “gene therapy” procedures. The cDNA sequence is cloned in a transduction vector of viral origin (lentiviral, retroviral, adenoviral, etc.) and assembled into a viral particle, capable of specifically targeting tumor or tumor-associated cells, by means of specific surface binding proteins. The viral particle preparation is then produced in a GMP grade facility. This preparation can be either systemically or intratumorally delivered through one single or multiple injections. Tumor tissues transduced by the viral vector will express the proteins encoded by the sequences of the anti-Met antibody, or derivatives or fragments thereof thus providing an auto-inhibitory circuit.

In the following experimental data are provided; the experiments have been conducted using DN30 monoclonal antibody and/or derivatives and/or fragments thereof in order to provide a detailed description of some preferred embodiments without any limiting purpose of the instant invention.

### Materials and Methods

Cell Lines and siRNA.

Cell lines (A549, MDA-MB-231, LoVo, MDAMB-4355, U-87MG, U-251, PC3, SF295, DLD1, SK-N-SH) were from ATCC. For ATM kinase inhibition, cells were pre-treated for 4 h before irradiation and then kept in the presence of CGK733 (10 μM in DMSO). siRNAs targeting RELA (ON-TARGET plus SMART pool L-003533-00 Human RELA, NM 021975, Dharmacon, 100 nM), or control siRNAs (ON-TARGET plus SMART pool, siCONTROL Non Targeting siRNA, Dharmacon) were transiently transfected.

The siRNA sequences were as follows.

“SMART pool L-003533-00 Human RELA NM 021975” was a 1:1:1:1: mixture of the following duplex sequences:

(SEQ ID No.: 1)
(1) sense: GGAUUGAGGAGAAACGUAAUU,
(SEQ ID No.: 2)
antisense: 5′-NUUUCCUACAAGCUCGUGGGUU,
(SEQ ID No.: 3)
(2) sense: CCCACGAGCUUGUAGGAAAUU,
(SEQ ID No.: 4)
antisense: 5′-NUUUCCUACAAGCUCGUGGGUU,
(SEQ ID No.: 5)
(3) sense: GGCUAUAACUCGCCUAGUGUU,
(SEQ ID No.: 6)
antisense: 5′-NCACUAGGCGAGUUAUAGCCUU,
(SEQ ID No.: 7)
(4) sense: CCACACAACUGAGCCCAUGUU,
(SEQ ID No.: 8)
antisense: 5′-NCAUGGGCUCAGUUGUGUGGUU.
SMART pool, siCONTROL Non Targeting siRNA
(one single duplex sequence):
(SEQ ID No.: 9)
sense: AUGUAUUGGCCUGUAUUAG.

DN30 Antibody and DN30 Fab Fragment Production.

DN30 monoclonal antibody was produced as described in Prat M. et al., 1998, J. Cell Sci 111:237-247, and deposited by Advanced Biotechnology Center with accession number ICLC PD 05006. The DN30 Fab fragment was obtained through DN30 papain digestion and affinity purification (Immunopure Fab Preparation Kit, Pierce). The aminoacid sequence of DN30 heavy chain is illustrated in FIG. 8b and set forth in SEQ ID No:10, DN30 heavy chain nucleotide sequence is illustrated in FIG. 8a and set forth in SEQ ID No.:11.

The aminoacid sequences corresponding to DN30 heavy chain CDR regions are the following: CDR-H1: GYTFTSYW (SEQ ID NO.:12); CDR-H2: INPSSGRT (SEQ ID NO.:13); CDR-H3: ASRGY (SEQ ID NO.:14). The nucleotide sequences corresponding to DN30 heavy chain CDR regions are the following: CDR-H1: GGCTACACCTTCACCAGTTACTGGA (SEQ ID NO.:15); CDR-H2: ATTAATCCTAGCAGCGGTCGTACT (SEQ ID NO.:16); CDR-H3: GCAAGTAGG (SEQ ID NO.:17).

The aminoacid sequence of DN30 light chain is illustrated in FIG. 9b and set forth in SEQ ID No:18, DN30 light chain nucleotide sequence is illustrated in FIG. 9a and set forth in SEQ ID No.:19.

The aminoacid sequences corresponding to DN30 light chain CDR regions are the following: CDR-L1: QSVDYDGGSY (SEQ ID NO.:20); CDR-L2: AAS (SEQ ID NO.:21); CDR-L3: QQSYEDPLT (SEQ ID NO.:22). The nucleotide sequences corresponding to DN30 light chain CDR regions are the following: CDR-L1: AAAGTGTTGATTATGATGGTGGTAGTTATAT (SEQ ID NO.:23); CDR-L2: GCTGCATCC (SEQ ID NO.:24); CDR-L3: CAGCAAAGTTATGAGGATCCGCTCACG (SEQ ID NO.:25).

In Vitro Irradiation.

X-rays were emitted by a linear particle accelerator (Clinac 600C/D, Varian) operating at 6 MV.

Western Blot.

Protein expression was investigated in irradiated confluent, serum-starved cells. For fractionation in cytoplasmic and nuclear portions, cells were washed and incubated on ice for 10 min in “buffer A” (10 mM HEPES pH 7.9, 10 mM KCl, 0.1 mM EDTA, 0.5% NP-40, 1 mM dithiothreitol, 1 mM phenylmethylsulfonyl fluoride and a cocktail of protease inhibitors). Supernatants, containing the cytoplasmic extracts, were separated by centrifugation. Pellets were resuspended in “buffer B” (20 mM HEPES pH 7.9, 400 mM KCl, 1 mM EDTA, 1 mM dithiothreitol, 10% glycerol, 1 mM phenylmethylsulfonyl fluoride and a cocktail of protease inhibitors) and incubated at 4° C. for 1 h with vigorous mixing. The resulting nuclear lysates were clarified by high-speed centrifugation. Equal amount of proteins were resolved by SDS-PAGE and analysed by western blot with the following primary antibodies: mouse anti-human Met (DL21 disclosed in Prat et al., Int. J. Cancer 49, 323-328 (1991)), mouse anti-p65/RelA and mouse anti-HIF-1α (BD Biosciences), rabbit anti-phospho-Ser276 and rabbit anticaspase-3 (Cell Signaling), rabbit anti-phospho-Chk2 (T68, R&D Systems). Goat anti-actin (C-11; Santa Cruz Biotechnology) and mouse anti-lamin B (Calbiochem) were used for control of equal cytoplasmic or nuclear protein loading, respectively. Blot images were captured using a molecular imager (GelDoc XR; Bio-Rad Laboratories). Densitometric analysis was performed with Quantity One 1-D (Bio-Rad Laboratories). Western blots shown are representative of results obtained in at least three independent experiments.

Northern Blot.

Confluent cells were serum-starved for 48 h and irradiated. Total RNA was resolved in 0.8% agarose-formaldehyde gel, and transferred to nylon membranes (Amersham) according to standard procedures. The MET probe containing the whole coding sequence (GenBank Accession N. J02958) was obtained from the pCEV-MET plasmid (see Michieli et al., Oncogene 18, 5221-5231 (1999)), and labelled with [α-³²P] dCTP (Megaprime, Amersham). Hybridization was carried out at 42° C. for 16 h in the presence of 50% formamide. Nylon membranes were washed twice with 2×SSC-0.1% SDS, and twice with 0.1×SSC-0.1% SDS at 42° C., and autoradiographed.

ROS Detection.

Intracellular ROS generation was assessed using 5-(and 6)-carboxy-2′-7′-dichlorofluorescein diacetate (carboxy-H₂DCFDA, Molecular Probes) according to manufacturer's instructions. Briefly, cells were seeded in black 96-well plates (3×10⁴ cells/well) 24 h before treatment (IR: 10 Gy; H₂O₂: 100 μM as control). Cells were incubated in the presence of carboxy-H₂DCFDA (10 μM) in phenol red-free DMEM for 1 h at 37° C. Cells were washed, incubated for additional 30 min in phenol red-free DMEM without carboxy-H₂DCFDA, and then irradiated. ROS generation was measured 15 min after irradiation using a fluorescence plate reader (λₑₓ=485 nm, λₑₘ=535 nm) (DTX 880 Multimode Detector).

Chromatin Immunoprecipitation (ChIP).

4×10⁷ cells were used for 10 ChIPs either for irradiated or control cells. After irradiation (10 Gy), cells were fixed in 1% formaldehyde for 15 min at room temperature, and reaction was stopped with glycine (0.125 M). Fixed cells were washed, collected in ice cold PBS supplemented with a cocktail of protease inhibitors and centrifuged. The cytoplasmic fraction was extracted as above and discarded, and nuclei were pelletted and resuspended in 1 ml of SDS-Lysis Buffer (1% SDS, 1 mM EDTA, 50 mM Tris-HCl pH 8, and a cocktail of protease inhibitors). Nuclei were then disrupted by sonication, yielding DNA fragments with a bulk size of 400-1000 bp. Cell debris were cleared by centrifugation at 14.000 rpm for 10 min at 4° C. The supernatants containing the chromatin preparation were diluted with Dilution Buffer 10× (0.01% SDS, 1.1% Triton X-100, 1.2 mM EDTA, 16.7 mM Tris-HCl pH 8, 167 mM NaCl). Chromatin was then pre-cleared for 1 h at 4° C. by adding protein G-sepharose (Amersham, 50% gel slurry supplemented with 0.2 mg/ml of salmon sperm DNA, 0.1% BSA and 0.05% NaN₂). Beads were pelleted by a brief centrifugation at 4000 rpm at 4° C. and supernatants were collected. 3% of chromatin preparation was used as Input for ChIP normalisation. ChIP was performed overnight at 4° C. with 4 μg of antibodies (anti-p65/RelA, Santa Cruz; total mouse IgG, Chemicon), followed by incubation with 50 μl of protein G-sepharose beads for 3 h. Beads were washed sequentially on a rotating platform at 4° C. with the following solutions (10 min/each): twice with Low-Salt Buffer (0.1% SDS, 1% Triton X-100, 2 mM EDTA, 20 mM Tris-HCl pH 8, 150 mM NaCl), twice with High-Salt Buffer (0.1% SDS, 1% Triton X-100, 2 mM EDTA, 20 mM Tris-HCl pH 8, 500 mM NaCl), once with LiCl Buffer (0.25 M LiCl, 1% Deoxycholic Acid, 0.5% NP-40, 1 mM EDTA, 10 mM Tris-HCl pH 8), and twice with TE (10 mM Tris-HCl pH 8, 1 mM EDTA). ChIPs were eluted twice in EB (1% SDS, 0.1 M NaHCO₃) and kept overnight at 65° C. to reverse formaldehyde cross-linking. Treatment with RNase (50 μg/ml, 37° C. for 30 min) and Proteinase-K (500 μg/ml, 45° C. for 2 h) were performed. Each sample was purified by phenol/chloroform extraction and finally resuspended in 40 μl of sterile water. 2 μl of each sample were used as template for Real-Time PCR with SYBR GREEN PCR Master Mix (Applied Biosystems) on ABI PRISM 7900HT sequence detection system (Applied Biosystems).

Primers used were as follows:

NFKBIA (fw: GAACCCCAGCTCAGGGTTTAG - SEQ ID No.:
26; rev: GGGAATTTCCAAGCCAGTCA - SEQ ID No.: 27);
κB1 (fw: AGGCCCAGTGCCTTATTACCA - SEQ ID No.: 28;
rev: GCGGCCTGACTGGAGATTT - SEQ ID No.: 29);
κB2 (fw: GGGACTCAGTTTCTTTACCTGCAA - SEQ ID No.:
30; rev: GGGACTCAGTTTCTTTACCTGCAA - SEQ ID No.:
31).

Wound Healing Assay.

Cells were grown to confluence in 24-well plates, starved for 24 hr, and scratched with a plastic tip. Culture medium was replaced with fresh medium containing 1% FBS and the vehicle alone (DMSO), and immediately irradiated. After 24 h, cells were fixed with 11% glutaraldehyde (Sigma), and stained with crystal violet. Images were acquired with a Leica photocamera (Leica DFC320, Leica) connected with an inverted light microscope (DM IRB, Leica). Images are representative of results obtained in at least three independent experiments.

Transwell Assay.

Cell invasion was measured in Transwell™ chambers (BD Falcon). MDA-MB-231 and MDA-MB-435S cells (5×10⁵/transwell) were seeded on the filter upper sides coated with 20 μg/cm² of reconstituted Matrigel basement membrane (Collaborative Research). U-251 (10⁴/transwell) were seeded on filters coated with 50 μg/cm². Culture medium supplemented with 1% FBS was added to both chambers. 1 h after seeding, cells were irradiated (10 Gy) and incubated at 37° C. for 24 h. Cells on the filter upper side were mechanically removed, and those migrated onto the lower side were fixed, stained, and photographed as above. For quantification of cell invasion, ten fields per experimental condition were randomly selected and micrographed as above with a 10× objective. Morphometric analysis was performed using MetaMorph 7.1 software. Images are representative of at least three independent experiments.

Branching Morphogenesis Assay.

MDA-MB-4355 spheroids were preformed by single-cell resuspension in 240 mg/ml methylcellulose (Sigma) and culture in nonadherent 96-well plates (Greiner) for 24 h. Spheroids were transferred into a matrix containing 1.3 mg/ml type I collagen from rat tail (BD Biosciences), 10% FBS, and 240 mg/ml methylcellulose. After 24 h, cells were irradiated and/or cultured in the presence of HGF for 7 days. HGF was obtained as a baculovirus recombinant protein in SF9 cells. The conditioned medium from uninfected cells was used as negative control. Images are representative of results obtained in three independent experiments.

Cell Viability Assay with DN30 Fab.

10³ cells were seeded in 96-well plates and grown for 24 h. Culture medium was replaced with medium containing 1% FBS and DN30 Fab (28 μg/ml) or the vehicle alone (PBS). After 24 h, cells were irradiated (10 Gy). Cell viability was assessed as above.

### Results

### IR Induces MET Transcription

The present inventors have previously shown that the MET proto-oncogene is transcriptionally regulated by extra- and intracellular specific signals, including growth factors and the oxygen sensor. Here it is investigated modulation of Met expression by exposure to therapeutic doses of IR (up to 10 Gy).

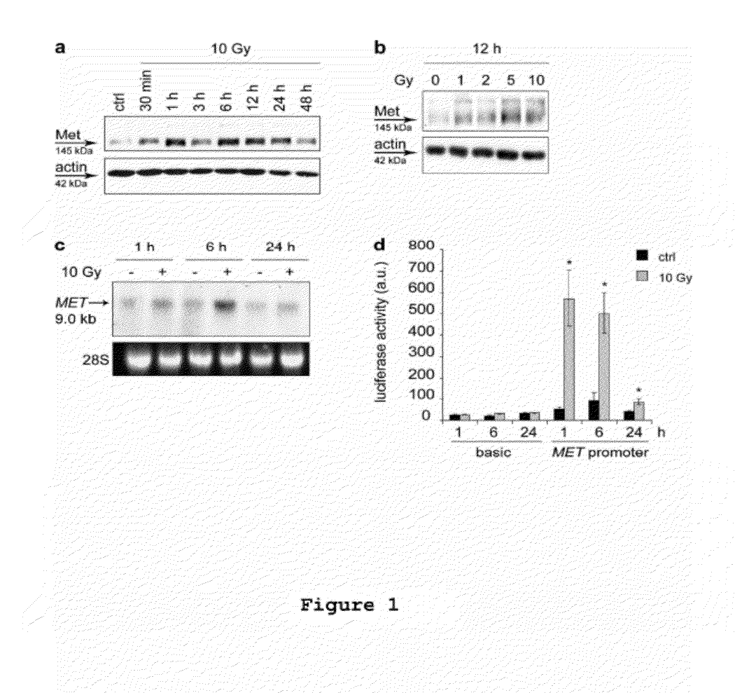

In ten cell lines derived from neoplastic tissues of different histological types (carcinomas of breast, lung, prostate and colon; melanoma; glioblastoma; neuroblastoma), it has been found that the Met protein level was significantly increased 24 h after irradiation. In representative cell lines (such as MDA-MB-4355 and MDA-MB-231), detailed time course experiments revealed a biphasic profile of Met protein accumulation. This is characterized by an early peak of Met induction (˜five fold) around 1-2 h, followed by a similar late peak or a plateau, appearing at 6 h, and decreasing 24 h after irradiation (FIG. 1a). Dose-response experiments showed that Met induction starts after 1 Gy, and reaches a plateau at 5 Gy (FIG. 1b). In irradiated cells, MET mRNA accumulation, and activation of the full-length MET promoter were also observed (FIG. 1c-d), indicating that IR-induced MET overexpression involves a transcriptional mechanism. Interestingly, in MDA-MB-231, a transient and ligand-independent Met autophosphorylation was also detected, occurring within 10 min after exposure to IR, (FIG. 6). The intensity of IR-induced Met phosphorylation was comparable to that elicited by a non-saturating concentration of HGF (50 ng/ml). However, the kinetics of phosphorylation were different, as the peak induced by IR was reached after 10 min, while the peak induced by HGF was reached after 30 min. Concomitant stimulation by IR and HGF was not synergistic (FIG. 6).

### IR-Induced MET Transcription Requires NF-κB

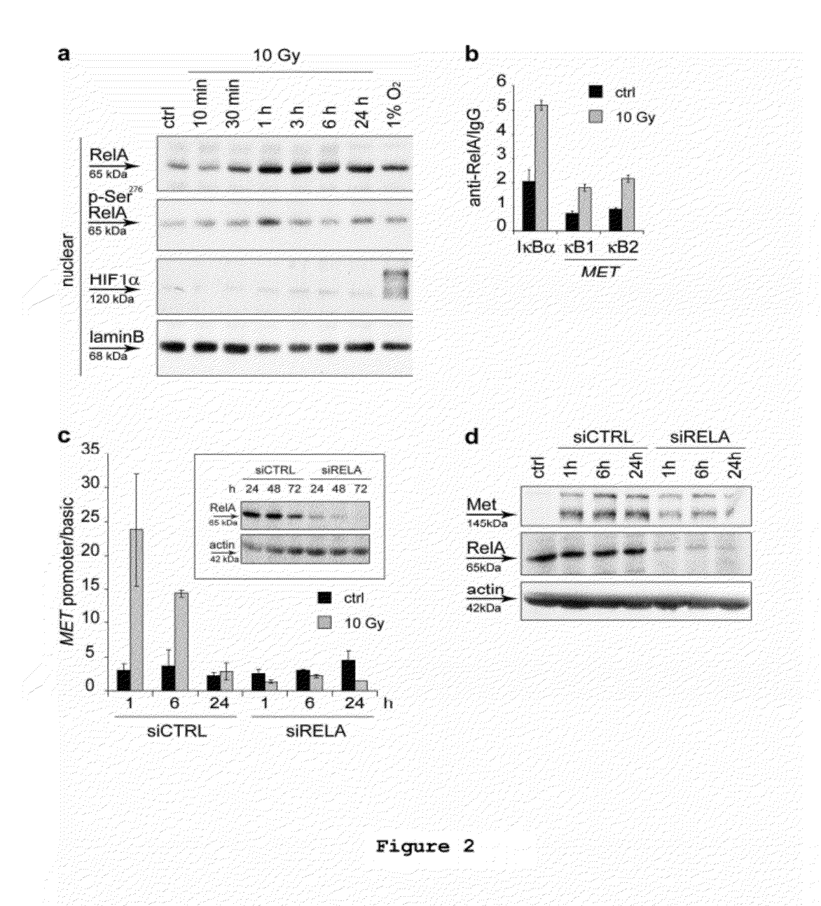

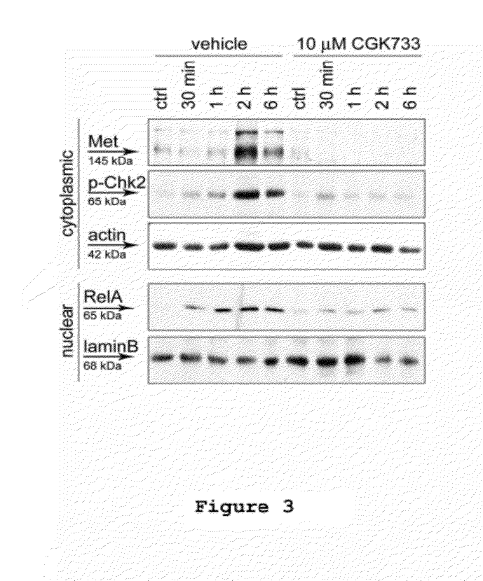

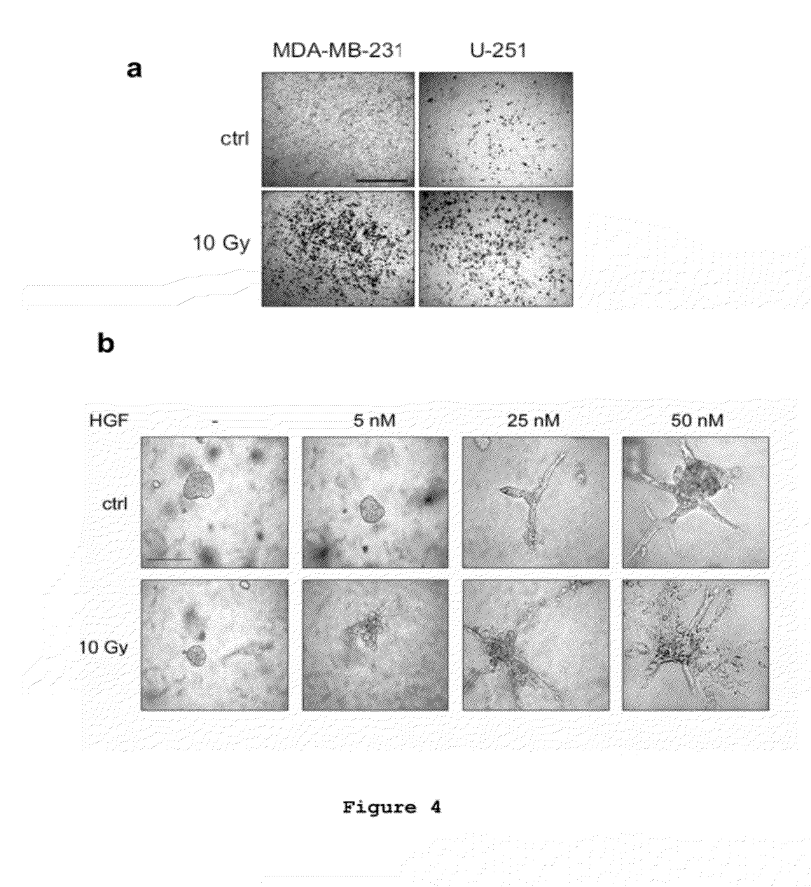

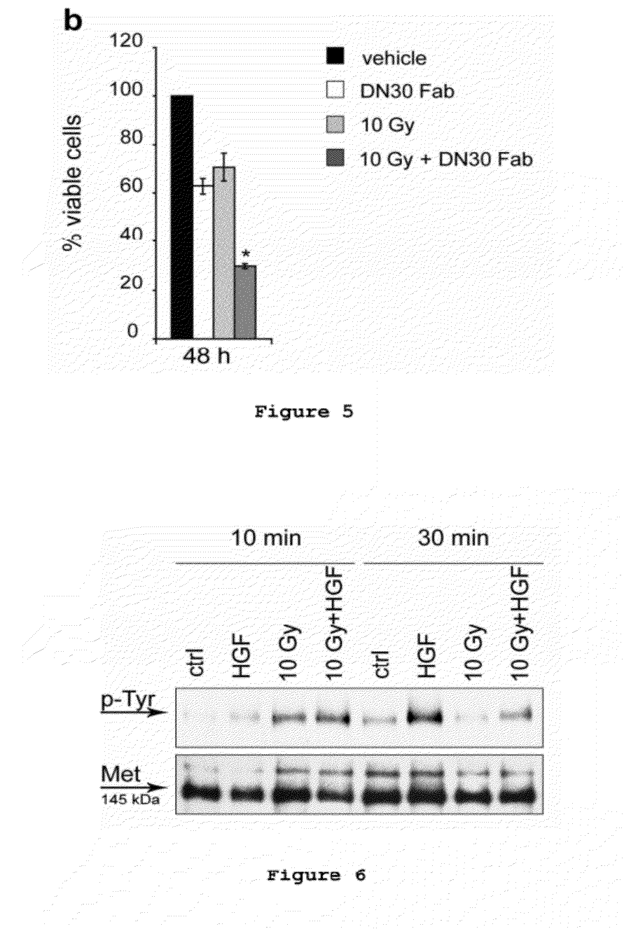

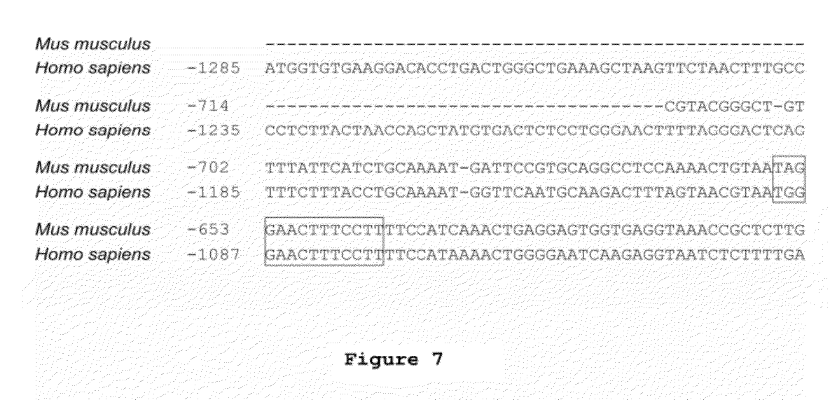

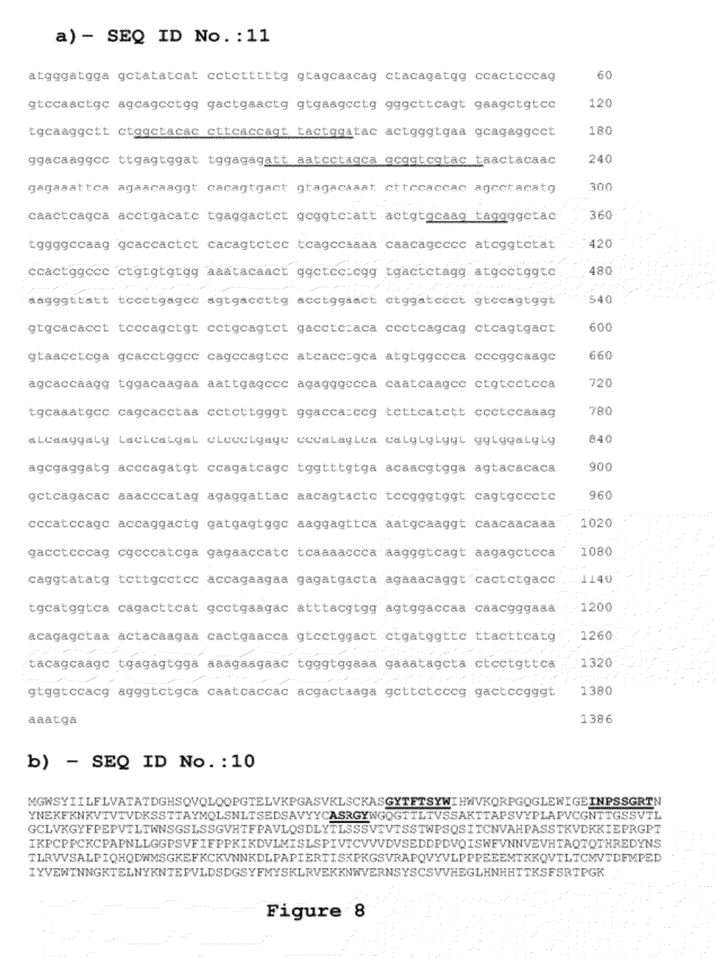

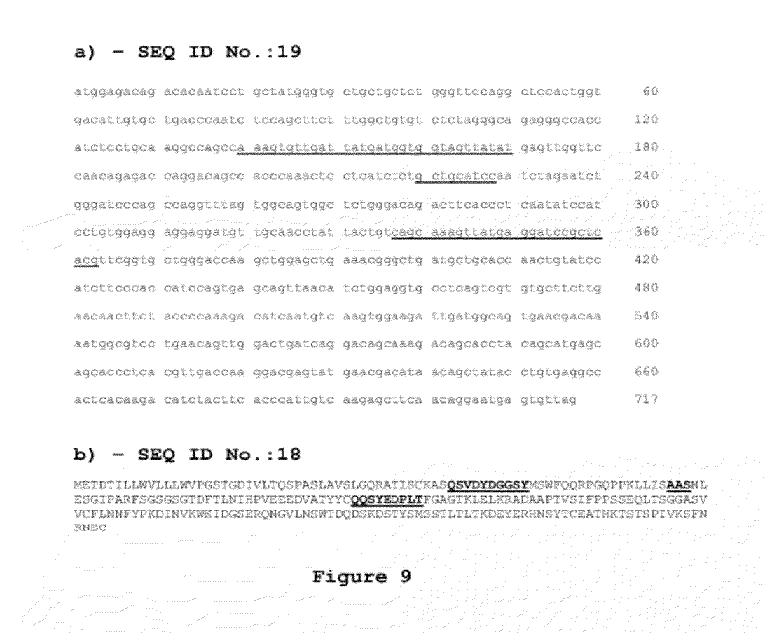

IR is known to modulate a few transcription factors including NF-κB. Accordingly, genome-wide expression profiling showed that, in the cell lines examined, IR induces a prominent early NF-κB response. For instance, in MDA-MB-231, 9 out of the 33 genes modulated 1 h after irradiation are NF-κB targets, displaying a frequency ˜20 fold higher than expected. Moreover, in time-course experiments with MDA-MB-231, MDA-MB-435S or U-251 cells, IR (10 Gy) induced rapid (within 30 min) and persistent (until 24 h) nuclear accumulation of the NF-κB subunit p65/RelA, a hallmark of NF-κB activation (FIG. 2a). Moreover, at early time-points after irradiation, nuclear p65/RelA was transiently phosphorylated at Ser²⁷⁶ (FIG. 2a). This phosphorylation is known to be induced by Reactive Oxygen Species (ROS) via protein kinase A, and to promote p65/RelA interaction with the transcriptional coactivator CBP/p300, which is required for upregulation of a subset of early target genes. These data indicate that IR promotes functional activation of NF-κB, through nuclear accumulation and early transient phosphorylation of the p65/RelA subunit.

In the MET human promoter, two putative NF-κB binding sites, κB1, located at −1349/−1340 bp, and κB2, located at −1149/−1136 bp, with respect to the transcription start site of the sequence (GenBank accession No. AF046925) were identified through in silico analysis. Interestingly, the κB2 site is highly conserved in the met mouse promoter (FIG. 7; met mouse (mus musculus) promoter sequence set forth in SEQ ID No:32 and met human (homo sapiens) promoter sequence set forth in SEQ ID No.:33). Chromatin immunoprecipitation experiments showed that association of p65/RelA to either site was significantly increased in cells exposed to 10 Gy (FIG. 2b), indicating that MET is transcriptionally controlled by NF-κB in irradiated cells. These findings prompted the present inventors to investigate whether NF-κB is an absolute requirement for MET induction by IR. As p65/RelA is involved in the formation of each of the several NF-κB heterodimers, thus being critical for the entire NF-κB-driven transcriptional activity, p65/RelA expression was abrogated through RNA interference. In MDA-MB-231 or MDA-MB-4355 treated with siRNA against p65/RelA (SMART pool L-003533-00 Human RELA, SEQ ID No.: 1 to 8), IR could no longer induce the full-length MET promoter activity (FIG. 2c), neither accumulation of the Met protein. Taken together, these data provide convincing evidence that IR-induced MET upregulation requires activation of the transcription factor NF-κB.

The involvement of Hypoxia Inducible Factor-1 (HIF-1) in IR-induced MET transcription was also considered, since (a) HIF-1 was shown to be activated in irradiated cells as result of ROS formation, and (b) HIF-1 is a prominent regulator of MET expression. However, the relevance of HIF-1 was minimal, as shown by complementary approaches. First, in MDA-MB-231 and MDA-MB-435S, IR did not induce nuclear translocation of the HIF-1α subunit, which is the hallmark of HIF-1 activation, otherwise observed when cells were cultured in low oxygen concentration (FIG. 2a). Lack of HIF-1 activation was not due to weak ROS production in irradiated cells, as ROS were increased by 25±3.5% on average, 15 min after exposure to 10 Gy. This was estimated to correspond to an average 80% ROS induction 2-5 min after irradiation, accordingly to previous observations in cell lines exposed to 1-10 Gy. Moreover, it has been found that IR could not activate the so-called “minimal” MET promoter including the two functional Hypoxia Responsive Elements (HRE), and the Ap-1 site, which are responsible for hypoxia-induced MET upregulation. Taken together, these data indicate that HIF-1 is not involved in MET upregulation by IR. However, it has been observed that hypoxia induced p65/RelA nuclear translocation and serine phosphorylation (FIG. 2a). Finally, the involvement of the transcription factor p53, a prominent IR-target, was ruled out as well. In fact, MDA-MB-435S and MDA-MB-231 (two cell lines displaying the highest MET induction by IR) harbour p53 inactivating mutations (G266E and R280K, respectively). Moreover, unlike the mouse promoter, the human MET promoter is not upregulated by constitutively active forms of p53.

### IR-Induced MET Expression is Mediated by ATM Kinase Activation

NF-κB is a crossroad of several pathways initiated both by extracellular and intracellular signals. The latter include those elicited by protein kinase ATM following detection of DNA damage. To investigate whether MET induction by IR relies on activation of the ATM kinase, MDA-MB-435S or MDA-MB-231 were treated with 10 μM of the specific small-molecule inhibitor CGK733. In time course experiments, CGK733 prevented IR-induced phosphorylation of the specific ATM substrate Chk2, as well as p65/RelA nuclear translocation, and Met protein overexpression. These data indicate that ATM kinase is required for IR-induced MET upregulation (FIG. 3).

### IR-Induced Invasive Growth Requires Met

Met overexpression does not imply kinase activation in the absence of the extracellular ligand HGF. However, it entails a significant increase in ligand-dependent signalling activity (i.e. sensitization). This has been observed in cells where hypoxia upregulated Met expression to a level comparable to, or lower than that induced by irradiation.

The present inventors thus investigated whether IR-induced Met overexpression could elicit or potentiate the Met-dependent biological responses. These include the physiological and pathological sides of invasive growth. In wound-healing assay, assessing the ability of the cell to regenerate injured tissues (i.e. physiological invasive growth), irradiated MDA-MB-231, as well as MDA-MB-435S, spontaneously performed the healing program, by detaching from the edge of the wound, and migrating throughout the scratched area. This response, monitored for 24 h, was overlapping with that stimulated by HGF, which is also known as “Scatter Factor”, as it promotes cell dissociation and motility. However, the healing response elicited by IR was not due to induction of a HGF autocrine loop, as irradiated cells did not express HGF as assessed by quantitative PCR. The present inventors conclude that IR-induced Met overexpression sensitizes cells to the small amount of HGF present in the culture medium, which was supplied with 1% serum. This condition likely mimics the physiological presence of HGF in vivo, which is ubiquitously embedded in extracellular matrices.

Irradiated cells were then assessed in transwell assays, measuring the ability to trespass an artificial basement membrane in vitro, which tightly correlates with in vivo invasiveness, i.e. malignant invasive growth. Indeed, irradiated cells (such as MDA-MB-231, MDA-MB-435S, or U-251) spontaneously crossed the transwell basement membrane in the presence of a low serum concentration (1%) (FIG. 4a), again mimicking the behaviour evoked by HGF.

IR Turns Met-Induced Morphogenesis into an Invasive Process

Branching morphogenesis is a complex physiological process, induced by HGF as to generate tridimensional organs during development. This multistep program entails cell migration, proliferation and spatial reorganization, ending up with generation of hollow branched tubules lined by polarized cells. Some of the cell lines studied, such as MDA-MB-4355, can fully execute the branching morphogenesis program in vitro.

Exposure to IR sensitized these cells to a suboptimal concentration of exogenous HGF (5 nM) that—alone—is incapable of inducing branching morphogenesis (FIG. 4b). Importantly, irradiated cells stimulated with HGF built tubules with remarkable structural alterations, as cells disengaged from the abluminal surface and spread into the surrounding matrix (FIG. 4b). This behaviour is reminiscent of the “tridimensional scatter” described as a form of aberrant morphogenesis occurring in response to TNFα. It was concluded that therapeutic doses of IR may turn physiological branching morphogenesis into an aberrant pro-invasive process.

### Met Inhibition Sensitizes Cells to IR-Induced Apoptosis and Proliferative Arrest

As part of the EMT/IG program, Met emanates powerful anti-apoptotic signals through sustained activation of downstream pathways including PI3-kinase/AKT. The present inventors thus reasoned that MET upregulation could prevent cell death induced by irradiation, and that, conversely, Met inhibition could increase the efficacy of radiotherapy.

A cell viability decrease (up to 75%) was observed in irradiated cells that were kept in the presence of the Fab fragment of the DN30 anti-Met antibody, which is known to induce MET down-regulation, thus inhibiting MET signalling and biological activities (Petrelli et al., PNAS 103: 5090-5, 2006) (FIG. 5).

These results indicate that Met inhibition activity sensitizes cells to radiotherapy, by increasing cell death and reducing the ability to resume proliferation after treatment.

Naturally, while the principle of the invention remains the same, the details of construction and the embodiments may widely vary with respect to what has been described and illustrated purely by way of example, without departing from the scope of the present invention.

1-13. (canceled)

14. A method of treating a patient suffering from a tumor comprising administering to said patient a Met inhibitor, said Met inhibitor being selected from the group consisting of:
i) an anti-Met monoclonal antibody,
ii) a genetically engineered antibody comprising the complementarity determining regions (CDRs) of the anti-Met monoclonal antibody, and
iii) a fragment of (i) or (ii) comprising the complementarity determining regions (CDRs) of the anti-Met monoclonal antibody, or combinations thereof,
wherein said Met inhibitor is able to induce down-regulation of the receptor encoded by the MET gene and reduces and/or abrogates patient's resistance to radiotherapy.

15. The method according to claim 14, wherein said Met inhibitor is selected from the group consisting of:
i) DN30 anti-Met monoclonal antibody,
ii) a genetically engineered antibody comprising the complementarity determining regions (CDRs) of DN30 anti-Met monoclonal antibody, said CDRs having the amino acid sequences set forth in SEQ ID NO.: 12 to 14 and 20 to 22, and
iii) a fragment of (i) or (ii) comprising the complementarity determining regions (CDRs) of DN30 anti-Met monoclonal antibody, said CDRs having the amino acid sequences set forth in SEQ ID NO.: 12 to 14 and 20 to 22, or combinations thereof,
wherein said DN30 anti-Met monoclonal antibody is produced by the hybridoma cell line ICLC PD 05006.

16. The method according to claim 14, wherein said Met inhibitor is administered in the form of soluble protein by injection or infusion.

17. The method according to claim 14, wherein said fragment is a Fab fragment, preferably a Fab fragment comprising at least one stabilizing molecule.

18. The method according to claim 17, wherein said at least one stabilizing molecule is selected from the group consisting of polyethylenglycol, albumin binding domain, and albumin.

19. The method according to claim 14, wherein said Met inhibitor is administered at least one week before subjecting said patient to radiotherapy.

20. The method according to claim 14, wherein said Met inhibitor is administered one day before subjecting said patient to radiotherapy.

21. The method according to claim 14, wherein said Met inhibitor is administered up to one week, preferably 6 to 48 hours, after the end of radiotherapy.

22. The method according to claim 14, wherein said tumor is selected from the group consisting of a carcinoma, a musculoskeletal sarcoma, a soft tissue sarcoma, a hematopoietic malignancy, a brain tumor, melanoma, mesothelioma, and Wilms' tumor.

23. A method of treating a patient suffering from a tumor comprising administering to said patient a nucleotide sequence encoding a Met inhibitor, said Met inhibitor being selected from the group consisting of:
i) an anti-Met monoclonal antibody,
ii) a genetically engineered antibody comprising the complementarity determining regions (CDRs) of the anti-Met monoclonal antibody, and
iii) a fragment of (i) or (ii) comprising the complementarity determining regions (CDRs) of the anti-Met monoclonal antibody,
wherein said Met inhibitor is able to induce down-regulation of the receptor encoded by the MET gene and reduces and/or abrogates patient's resistance to radiotherapy.

24. The method according to claim 23, wherein said Met inhibitor is selected from the group consisting of:
i) DN30 anti-Met monoclonal antibody,
ii) a genetically engineered antibody comprising the complementarity determining regions (CDRs) of DN30 anti-Met monoclonal antibody, said CDRs having the amino acid sequences set forth in SEQ ID NO.: 12 to 14 and 20 to 22, and
iii) a fragment of (i) or (ii) comprising the complementarity determining regions (CDRs) of DN30 anti-Met monoclonal antibody, said CDRs having the amino acid sequences set forth in SEQ ID NO.: 12 to 14 and 20 to 22, or combinations thereof,
wherein said DN30 anti-Met monoclonal antibody is produced by the hybridoma cell line ICLC PD 05006.

25. The method according to claim 23, wherein said nucleotide sequence encoding said Met inhibitor is administered by means of a vector, wherein said vector is in the form of a particle.

26. The method according to claim 25, wherein said vector is suitable for targeting tumor or tumor-associated cells.

27. The method according to claim 25, wherein said vector is for systemic or intra-tumor administration, preferably by injection.

28. The method according to claim 23, wherein said fragment is a Fab fragment, preferably a Fab fragment comprising at least one stabilizing molecule.

29. The method according to claim 28, wherein said at least one stabilizing molecule is selected from the group consisting of polyethylenglycol, albumin binding domain, and albumin.

30. The method according to claim 23, wherein said nucleotide sequence encoding said Met inhibitor is administered at least one week before subjecting said patient to radiotherapy.

31. The method according to claim 23, wherein said nucleotide sequence encoding said Met inhibitor is administered one day before subjecting said patient to radiotherapy.

32. The method according to claim 23, wherein said nucleotide sequence encoding said Met inhibitor is administered up to one week, preferably 6 to 48 hours, after the end of radiotherapy.

33. The method according to claim 23, wherein said tumor is selected from the group consisting of a carcinoma, a musculoskeletal sarcoma, a soft tissue sarcoma, a hematopoietic malignancy, a brain tumor, melanoma, mesothelioma, and Wilms' tumor.
